(19) United States
(12) Patent Application Publication
(10) Pub. No.: US 2005/0031671 A1
(43) Pub. Date: Feb. 10, 2005

(54) Weight management, longevity and health paradigm

(76) Inventor: James Johnson, Metairie, LA (US)

Correspondence Address: DRINKER BIDDLE & REATH, ONE LOGAN SQUARE, 18TH AND CHERRY STREETS, PHILADELPHIA, PA, 19103-6996, US

(21) Appl. No.: 10/844,535

(22) Filed: May 13, 2004

Related U.S. Application Data
(60) Provisional application No. 60/492,730, filed on Aug. 6, 2003.

(51) Int. Cl. A61K 47/00; A61B 5/00

(52) U.S. Cl. 424/439; 600/300

(57) ABSTRACT

Weight management predicated on intermittent caloric intake is provided. In an illustrative implementation, a selected percentage of a total caloric intake is provided and consumed over a first time period T1 and a second percentage of the total caloric intake is provided and consumed over a second time period T2 such that the average of the caloric intake over time periods T1 and T1 falls within a selected percentage range of the total caloric intake. Weight management subjects repeatedly intake the first and second selected percentages of the total caloric intake during the selected time periods T1 and T2 for a period of time T3 or until certain weight management goals have been achieved or other health benefits or relief of disease symptoms is achieved. Additionally, weight management subjects chronicle their caloric intake in a journal or other memorializing media during time period T3 or until certain weight management goals have been achieved.

### CLAIM OF PRIORITY

This application claims the benefit of and priority to, U.S. Provisional Application 60/492,730, filed on Aug. 6, 2003, entitled, “PROCESS FOR WEIGHT CONTROL AND LONGEVITY EXTENSION THROUGH DIETARY MANAGEMENT,” which is herein incorporated by reference in its entirety.

### FIELD OF THE INVENTION

The invention relates to managing body weight, and more particularly, to a weight management paradigm employing temporal based caloric restrictions that also contributes to the longevity of the subject and overall health benefits.

### BACKGROUND OF THE INVENTION

The need to control food intake among Americans has become more critical year after year. Estimates are that the average weight for adults has increased 16 pounds or more in the past twenty years. The incidence of non insulin dependent diabetes mellitus has increased rapidly, such that projections are that 29% of children will develop non-insulin dependent diabetes mellitus (NIDDM) within 15 years.

It is conjectured that the success of any weight reduction diet is dependent on compliance. The factors which affect compliance include, but are not limited to, degree of hunger sensed and psychological factors related to the sense of despair, failure, self deprecation, stress of the diet itself, the type of food consumed, and taste. The psychological mechanisms of denial and rationalization are theorized to play a major role in failure to maintain adherence to the diet. Popular diets require considerable preparation and forethought on a daily or more frequent basis. This can be daunting for many, and especially over a long period of dieting. As with exercise, there is a presumption that the enjoyment of the process greatly affects willingness to stick with a particular diet. In addition, dieters often encounter a lack of energy and face a constant fear that he/she will be exposed to temptation, which only adds to a dieter's stress.

Many studies have been performed on animals since the 1930's demonstrating a consistent increase in lifespan by the use of calorie restriction, (CR). A wide range of species has been tested, including protozoa, insects, rodents, monkeys and others. A consistent pattern of effect has been demonstrated that, as a generalization, a 40% reduction in calories leads to a 40% increase in both the average and maximum lifespan for the species. The mechanism is fully not known, but it is theorized that it relates to one or more adaptations occurring early in the evolutionary process for a broad range of species. With CR, the tested animals show consistent weight loss and lower metabolic function over time. It is accepted that the Sir2 gene in animals (known as SIRT1 gene in humans) is activated by CR.

Data from such studies suggests that CR may contribute to extending both the average and maximum species' lifespan. Beneficial changes in physiology measured by biochemical markers are seen in CR animals. The humoral factors elaborated by the CR animal may account for at least some of the effects seen, which may be the result of the activation of possibly many different “longevity” genes. Among some beneficial effects seen from CR studies in animals are reduction in atherosclerosis, lower incidence of NIDDM, protection against renal disease, a lower incidence of cancer and protection of the nervous system from disease and injury. Chronic inflammatory processes are also diminished. The data further suggests that humans may also respond to CR like other species.

From the foregoing it is appreciated that there exists a need for a CR based weight management paradigm that overcomes the limitations of existing practices.

### SUMMARY

A system and method are provided for weight management predicated on intermittent caloric intake. In an illustrative implementation, a selected percentage of a total caloric intake is provided and consumed over a first time period T1 and a second percentage of the total caloric intake is provided and consumed over a second time period T2. The illustrative implementation further provides that time period T2 is selected to be greater than time period T1. Furthermore, in the exemplary implementation, the average of the caloric intake over time periods T1 and T2 falls within a selected percentage range of the total caloric intake.

In operation, a participating weight management subject repeatedly intakes the first and second selected percentages of the total caloric intake during the selected time periods T1 and T2 for a period of time T3 or until certain weight management goals have been achieved. Additionally, the illustrative implementation further provides that the participating weight management subject chronicles his/her caloric intake in a journal or other memorializing media during time period T3 or until certain weight management goals have been achieved. In addition, improvement in significant symptoms of a variety of diseases can be expected based on animal studies and may serve as a guide to continuing the pattern of eating.

In an alternative illustrative implementation, the participating weight management subject repeatedly intakes the first and second selected percentages of the total caloric intake for a selected number of times during a time period T4 so to engage a weight maintenance mode.

Other features and aspects of the herein described systems and methods are further described below.

### BRIEF DESCRIPTION OF THE DRAWINGS

For the purpose of illustrating the invention, there is shown in the drawings illustrative implementations of the herein described systems and methods. It is appreciated that the herein described systems and methods are not limited to the precise arrangements and instrumentalities shown. The drawings are not necessarily to scale, emphasis instead being placed on illustrating the principles of the herein described systems and methods in which:

FIG. 1 is a block diagram showing a prior art illustrative weight management approach;

FIG. 2 is a block diagram showing a second prior art illustrative weight management approach;

FIG. 3 is a block diagram of an exemplary weight loss paradigm system and the cooperation of its components in accordance with the provided illustrative implementations;

FIG. 4 is a block diagram showing an illustrative implementation of a weight loss paradigm in accordance with the herein described systems and methods;

FIG. 5 is a block diagram showing a second illustrative implementation of a weight loss paradigm in accordance with the herein described systems and methods;

FIG. 6 is a flowchart showing the acts performed in accordance with the herein described illustrative implementation; and

FIG. 7 is a flowchart showing the acts performed in accordance with the herein described second illustrative implementation.

### DETAILED DESCRIPTION OF THE ILLUSTRATIVE IMPLEMENTATION

Overview:

Although difficult to maintain, calorie restrictive diets may provide significant health benefits outside of pure weight management. Specifically, data from recent studies indicate that in animal models, rats that were fed every other day (intermittent feeding) lived as long as a group of rats that were provided calorie restricted diets. Intermittently fed rodents usually show weight loss comparable to rodents subjected to chronic daily CR. Some strains of rodent will, however, maintain body weight comparable to control animals fed ad lib. The data from such studies as found in the article, “Intermittent fasting dissociates beneficial effects of dietary restriction on glucose metabolism and neuronal resistance to injury from calorie intake”, Anson, Michael R., et al., Proceedings of the National Academy of Sciences of the United States of America (PNAS), May 13, 2003, which is hereby incorporated by reference in its entirety, indicates that the weight of the mice fed intermittently was the same as the control group fed ad libitum, yet the intermittently fed mice showed profound beneficial physiologic changes. This and other studies suggest that a period of time of many hours during which no (or low) energy is available activates the mechinism.

The mechanism by which caloric restriction or intermittent fasting causes increase in lifespan is not entirely known, but it is conjectured and evidence has been noted in the article, “Small molecule activators of siruins extend Saccharomyces cerevisiae lifespan,” Howitz, Konrad T., et al., Nature 425, 191-196 (11 Sep. 2003); Nature AOP, published online 24 Aug. 2003, which is herein incorporated by reference in its entirety, that caloric restriction activates the enzyme Sir2 in Saccharomyces cerevisiae (brewer's yeast) and such activation may contribute to an extended lifespan of such organism. Similar tests have shown that the activation of the equivalent human enzyme, SIRT1, in cell culture enhances cell survival in the face of stresses such as ionizing radiation. It is further conjectured that SIRT1 activation gives injured cells extra time to repair themselves and survive longer.

As compared with calorie restrictive weight management approaches, commonly practiced weight management approaches impose significant food-type restrictions on their participating subjects. For example, in some approaches, participating subjects are relegated to eating protein rich foods foregoing foods having significant carbohydrates. Other approaches require that their subjects forgo all protein rich foods in favor of vegetables and fruits. Yet another approach requires their subjects to intake foods on a balanced basis (e.g. 6-12 servings of carbohydrates per day, 2-4 servings of protein per day, etc.).

The herein described systems and methods ameliorate the shortcomings of existing approaches by offering a weight management paradigm that does not place severe food restrictions as conventional weight management approaches whilst promoting the powerful benefits associated with calorie restriction. In an illustrative implementation, a modified fast is provided, the modified fast day being referred to as the “down” day, the normal eating day as the “up” day. In the illustrative implementation, the “down” day might consist of a period starting at midnight (12 A.M.) and ending at 6 A.M., 30 hours later. The “up” day might start at 6 A.M. and end at midnight (12 A.M.) 18 hours later. The duration of the total modified fast is extended by encouraging the participating weight management subject to wait as long as possible on the “up” day before he consumes any calories, and to not eat past 8 p.m. on the “up” day, thus lengthening the “down” day period to 36 hours or more.

Moreover, in the illustrative implementation, on the “up” day the weight management subject is encouraged to eat whatever he/she likes, and as much as he/she likes, but to keep a record of all food consumed, and to preferably not exceed his/her normal daily caloric requirement. Furthermore, the weight management subject may determine his/her normal daily caloric requirement from various food intake tables widely available from various sources. Weight reduction is usually the main goal of the paradigm described herein, and the subject will intake an average percentage below norm within a selected range (e.g. 50-70%) during the course of an “up” and “down” total time period. However, if the goal is treatment or prevention of disease or increased longevity, the subject may intake a low percentage of required maintenance in T1 and exceed in T2 100% of 24 hour maintenance by an amount which when added to T1 intake will equal 200% (or 100% per 24 hour period), thus producing weight equilibrium. Activation of the beneficial genetic CR mechanism is expected to occur.

It is appreciated that although the herein described systems and methods have been described to be performed during specific time periods and using specific percentages of caloric intake that such description is merely exemplary as the inventive concepts described herein may be applied over various time periods and using various percentages of caloric intake.

Prior Art Weight Management Approaches:

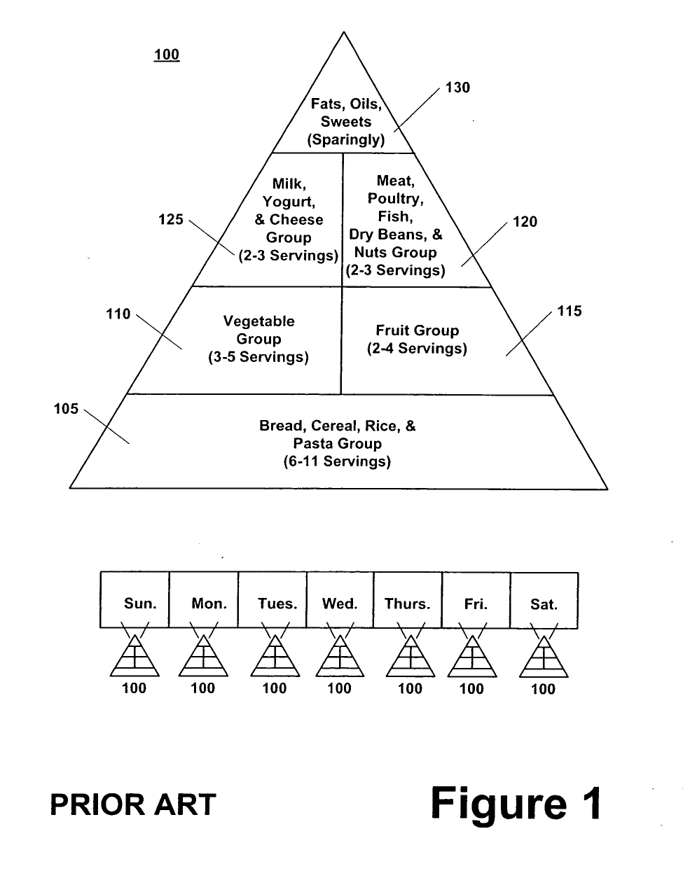

FIG. 1 shows a block diagram of a prior art weight management approach. The weight management approach is based on the United States Department of Agriculture's “Food Guide Pyramid” 100. Food Guide Pyramid (FGP) 100 provides an outline of what to eat each day. As is shown in FIG. 1, FGP 100 comprises several food type groups 105, 110, 115, 120, 125, and 130, each group providing a recommended daily serving amount. Food type group 105 comprises breads, cereals, and pasta groups having a recommended serving amount of 6-11 servings per day. Vegetable food group 110 recommends 3-5 servings of vegetables per day. Fruit group 115 recommends 2-4 servings of fruits per day. Food group 120 comprises meat, poultry, fish, dry beans and nuts having a recommended serving amount of 2-3 servings per day. Food group 125 comprises milk, yogurt and cheese having a recommended serving amount of 2-3 servings per day. Lastly, FGP 100 comprises food group 130 standing atop of FGP 100 which comprises fats, oils, and sweets. FGP 100 suggests that the foods found in food group 130 be eaten sparingly on a daily basis.

In practice, the foods found in FGP 100 are consumed according to the recommended serving amounts. The exact serving amounts and types of food within each food group varies according to the age, height, and weight of the participating weight management subject. Moreover, FGP 100 is put into practice from day to day, week to week, month to month, year to year, or until certain weight management goals and milestones have been achieved.

Although FGP 100 is not a rigid prescription, it provides weight management principals which, if followed, is purported to help participating subjects to achieve their desired weight management goals It is appreciated, however, that although extremely flexible in the amount and type of foods participating weight management subjects can intake, that such prior art weight management approach may be ineffective in triggering the above-described benefits derived from reduced calorie weight management approaches.

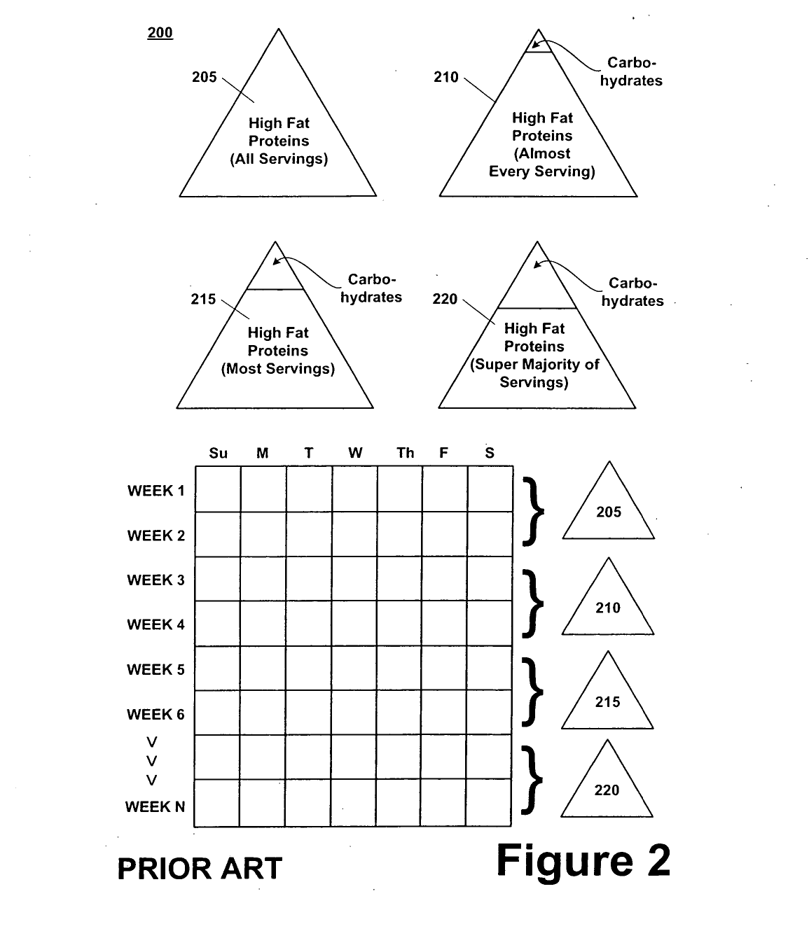

FIG. 2 shows a block diagram of a second prior art weight management approach. As is shown, prior art weight management approach 200 provides four recommended food intake triangles 205, 210, 215, and 220 that recommend the food types to be consumed for a particular meal or over a given day. The food types in food intake triangles 205, 210, 215, and 200, respectively, generally comprise high fat proteins, and carbohydrates in varying proportions. Specifically, food intake triangle 205 recommends the intake of high fat proteins exclusively. Comparatively, food intake triangle 210 recommends the intake of mostly high fats proteins (almost every serving) with very limited carbohydrate intake. Food intake triangle 215 is similar to food intake triangle 210, however recommending a slightly larger portion of carbohydrate intake (i.e. high fat proteins for most food intake servings). Lastly, food intake 220 triangle shows that carbohydrates account for a growing percentage of the total food intake.

In practice, weight management approach 200 recommends that the participating weight management subject intake a particular food intake triangle with its prescribed food type percentages for varying periods of time. For example, as is shown in the bottom of FIG. 2, weight management approach 200 recommends that food intake triangle 205 be consumed for the first two weeks of the weight management approach 200 program. After the first two weeks, participating weight management participants are asked to consume the foods in the proportions prescribed by food intake triangle 210 for the next period of time (e.g. week 3 and week 4). For the next time period (e.g. week 5 and week 6), weight management approach 200 suggests that participating users consume food in the proportions prescribed by food intake triangle 215. Lastly, the participating user is directed to consume food in the proportions prescribed by food triangle 220 for the remainder of the weight management approach 200 program time.

Commercial implementations of these weight management approaches include but are not limited to the ATKINS DIET and the SOUTH BEACH DIET. Such weight management approaches have been chronicled as effective in assisting participants to lose and/or maintain weight. It is appreciated, however, that weight management approach 200 places significant food-type restrictions on participating subjects which does not promote long term compliance and does not trigger the above-described benefits realized from calorie restriction.

Intermittent Feeding Weight Management Paradigm:

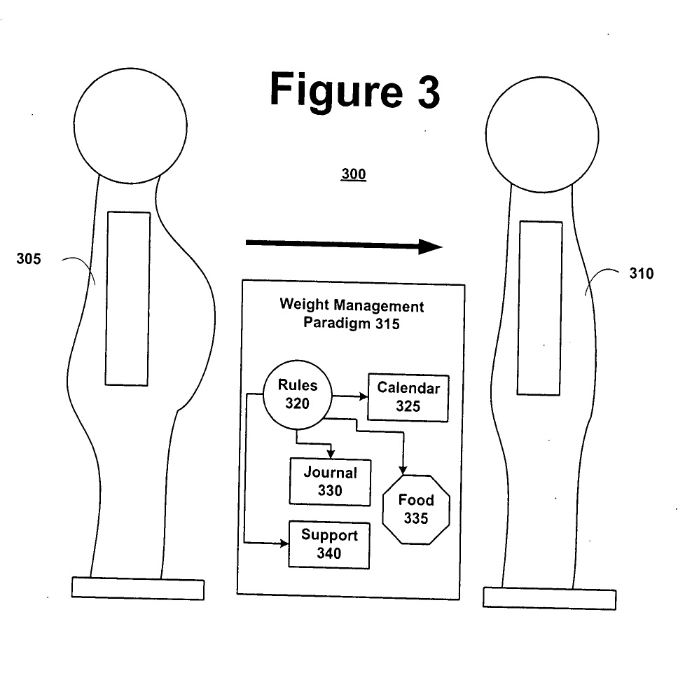

FIG. 3 shows the components of weight management environment 300 premised on calorie restricted intermittent feeding. As is shown, weight management environment 300 comprises a weight management participant being in a first weight state 305 and the weight management participant having a second weight state 310 after practicing weight management paradigm 315. First weight state 305 shows the weight management participant as being portly, wherein second weight state 310 shows the weight management participant having a more healthy body shape.

Furthermore, as is shown in FIG. 3, weight management paradigm 315 comprises rules 320, calendar 325, journal 330, food 335, and support 340. In operation, rules 320 provide the weight management participant instructions for using the calendar 325, journal 330, and intaking food 335. Additionally, rules 320 provide the weight management participant with guidance of how to use and rely on support 340.

In an illustrative implementation, rules 320 may provide the participating weight management user with instructions on how to consume food 335 according to selected calendar 325 and when to record the consumption of food 335 in journal 330. In this illustration, rules 320 may also provide guidance to the weight management participant regarding how to employ support so as to adhere to the consumption and time rules. Support 340 may include but is not limited to, food supplements (or replacements), psychological counseling, health counseling, and food consumption protocols.

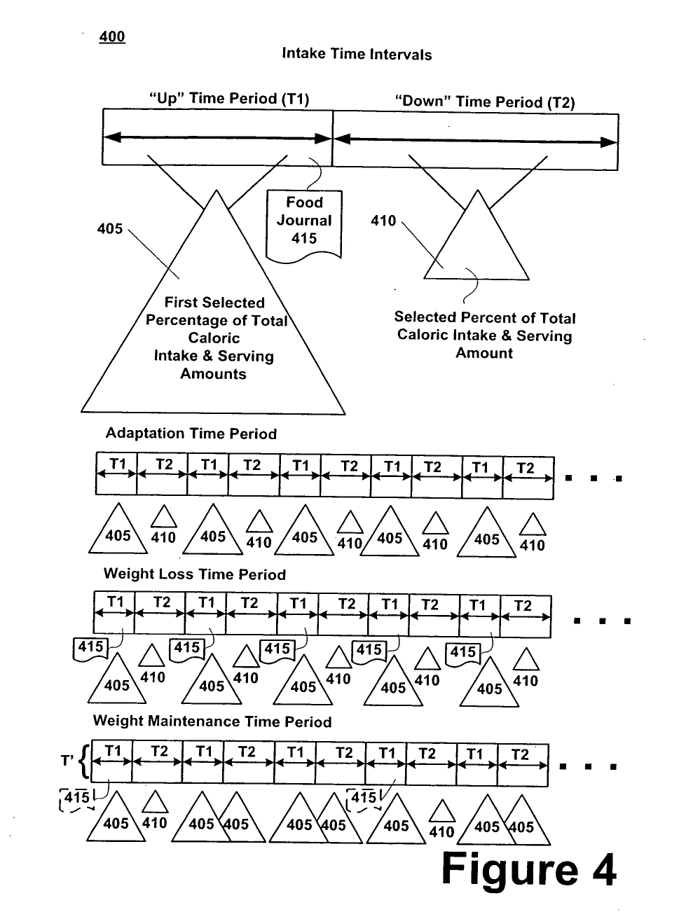

FIG. 4 shows the application of weight management paradigm 315. As is shown, weight management paradigm application 400 comprises intake time intervals T1 (“Up” time period) and T2 (“Down” timer period). Additionally, weight management paradigm application 400 further comprises food journal 415, first selected percentage of a total caloric intake and serving amounts 405 for consumption during time period T1, and a second selected percent of a total caloric intake and serving amount 410 for consumption during time period T2. Furthermore, as is shown in FIG. 4, weight management application 400 maintains several modes (portions). The portions described include adaptation time period, weight loss time period, and weight maintenance time period.

In operation, if the weight management participant (i.e. dieter—not shown) is engaged in either the adaptation time period or the weight loss time period, weight management paradigm application 400 directs the weight management participant (not shown) to consume the first selected percentage of the total caloric intake and servings 405 during time period T1 and the second selected percentage of the total caloric intake 410 during time period T2. As is further shown, such pattern is repeated by the participant (not shown) until the adaptation and/or weight loss time period have expired or until one or more weight management goals and milestones have been reached (as indicated by the elipses). During the weight loss time period, however, weight management paradigm application 400 directs the participant (not shown) to chronicle their food intake in journal 415 during each time period T1 during the duration of the weight loss time period.

Comparatively, during the weight maintenance time period, as is shown, weight management paradigm application 400 directs the weight management participant (not shown) to consume first selected percentage of the total caloric intake 405 during time period T1 and second selected percentage of the total caloric intake 410 during time period T2, and to repeat such practice for a selected number of times in a given time period T′. Furthermore, as is shown, for those time periods T1 and T2 in the weight maintenance time period T′ when not practicing the weight management paradigm, the participant (not shown) is directed to consume first selected percentage of the total caloric intake 405. As such, the participant (not shown) is consuming the first selected percentage of the total caloric intake more frequently. Additionally, similar to the weight loss portion and as is shown, weight management paradigm application 400 prescribes that the participant (not shown) chronicle the food intake in journal 415.

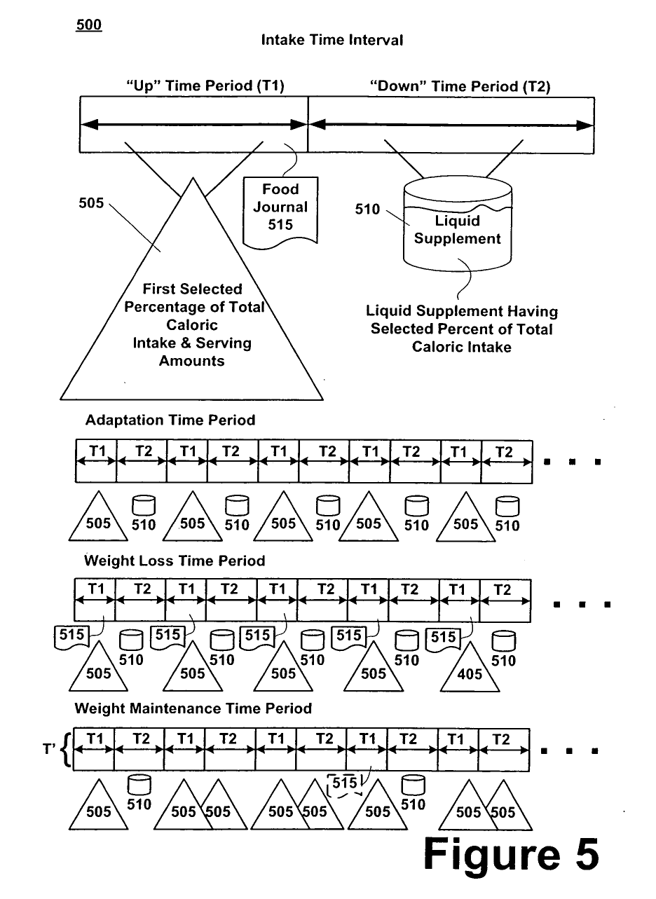

FIG. 5 shows a second illustrative application of weight management paradigm 315. As is shown, weight management paradigm application 500 comprises intake time intervals T1 (“Up” time period) and T2 (“Down” time period). Additionally, weight management paradigm application 500 further comprises food journal 515, first selected percentage of a total caloric intake and serving amounts 505 for consumption during time period T1, and a liquid supplement 510 having a second selected percent of a total caloric intake and serving amount for consumption during time period T2. Furthermore, as is shown in FIG. 5, weight management application 500 maintains several modes (portions). The portions described include adaptation time period, weight loss time period, and weigh maintenance time period.

In operation, if the weight management participant (i.e. dieter—not shown) is engaged in either the adaptation time period or the weight loss time period, weight management paradigm application 500 directs the weight management participant (not shown) to consume the first selected percentage of the total caloric intake and servings 505 during time period T1 and the liquid supplement 510 having a second selected percentage of the total caloric intake during time period T2. As is further shown, such pattern is repeated by the participant (not shown) until the adaptation and/or weight loss time period have expired or until one or more weight management goals and milestones have been reached. (as indicated by the elipses). During the weight loss time period, however, weight management paradigm application 500 directs the participant (not shown) to chronicle their food intake in journal 515 during each time period T1 during the duration of the weight loss time period.

Comparatively, during the weight maintenance time period, as is shown, weight management paradigm application 500 directs the weight management participant (not shown) to consume first selected percentage of the total caloric intake 505 during time period T1 and liquid supplement 510 having a second selected percentage of the total caloric intake during time period T2, and to repeat such practice for a selected number of times in a given time period T′. Furthermore, as is shown, for those time periods T1 and T2 in the weight maintenance time period T′ when not practicing the weight management paradigm, the participant (not shown) is directed to consume first selected percentage of the total caloric intake 505. As such, the participant (not shown) is consuming the first selected percentage of the total caloric intake more frequently. Additionally, similar to the weight loss portion and as is shown, weight management paradigm application 500 prescribes that the participant (not shown) chronicle the food intake in journal 515.

It is appreciated that the value for the total caloric intake as provided in FIGS. 4 and 5 may be obtained from conventional dietary guidelines including published guidelines from the United States Department of Agriculture. These guidelines prescribe the total amount of calories to consume in a given day. In the implementation provided, the inventive concepts described herein contemplate the selection of values for the first and selected percentages of the total caloric to be such that the average of the first and second selected percentages fall within a define percentage range. For example, in the implementation provided, the first selected percentage may be selected to have a value of 100% of a recommend daily caloric intake, wherein the second selected percentage may be selected from a range between 10 and 40% (must be less than 70% per day) of a recommended daily total caloric intake. Accordingly, the average percentage of caloric intake over the provided time periods T1 and T2 would fall within the range of 55-70%. Also, the values selected for time periods T1 and T2 are such that T2 is greater than T1. In an illustrative implementation, T1 may be a period of 18 hours, wherein T2 is a period of 30 hours. Lastly, when engaged in the weight maintenance mode, the participant is directed to perform a select number of fasts over a selected time period. In an illustrative implementation, to maintain weight, the participant may be directed to perform 2 “down” modified fast days (e.g. 60 hours) in a given week.

It is further appreciated that although the inventive concepts in FIGS. 4 and 5 have been described, by way of example, of having particular values that such values are merely exemplary since the inventive concepts described herein may apply to selected percentages having various values and ranges.

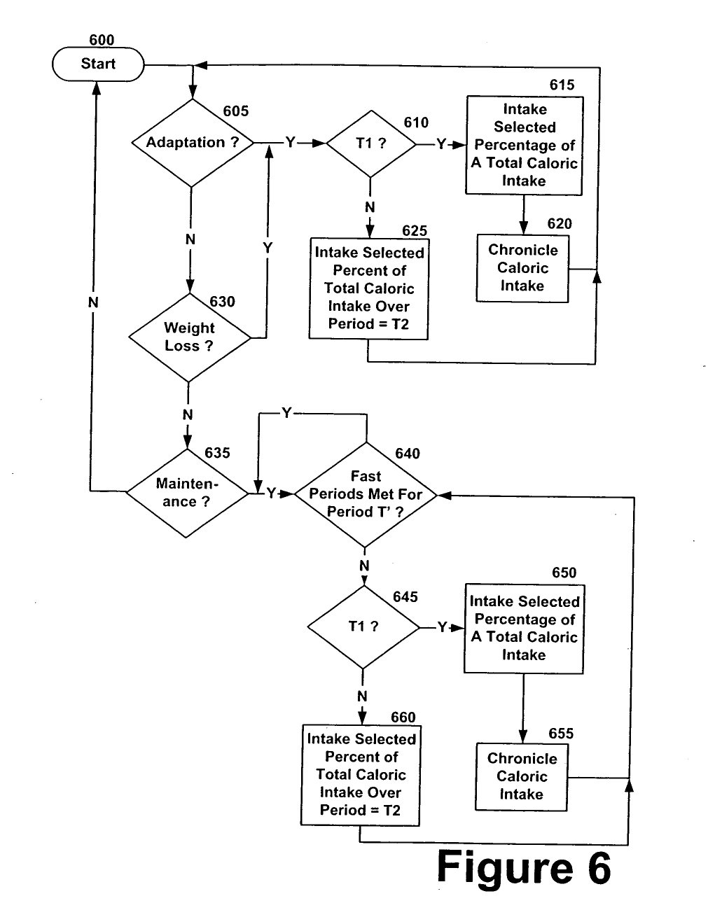

FIG. 6 shows the acts performed when applying weight management paradigm 315. As is shown, weight management paradigm 315 begins at block 600 and proceeds to block 605 where a check is performed to determine if the weight management participant is engaged in the adaptation portion of the weight management paradigm. If the weight management participant is engaged in the adaptation portion of the weight management paradigm, the paradigm proceeds to block 610 where a check is performed to determine if the weight management paradigm in within the selected time period T1. If the check at block 610 indicates that it is time period T1, the paradigm proceeds to block 615 where the participant is directed to consume a selected percentage of a total caloric intake. The paradigm then directs the participant to chronicle the caloric intake of block 615 at block 620. From there the weight management paradigm processing reverts to block 600 and continues onward.

If, however, the check at block 610 indicates that the paradigm is not within the selected time period T1, it is presumed that the paradigm is within the selected time period T2 (not shown) and the participant is directed to intake a second selected percentage of the total caloric intake over time period T2. From there, the paradigm reverts to block 600 and continues onward.

In the instance, however, it is determined that at block 605, the weight management participant is not engaged in the adaptation portion of the weight management paradigm, the paradigm proceeds to block 630 where a check is performed to determine if the weight management participant is engaged in the weight loss portion of the weight management paradigm. If the check at block 630 indicates that the participant is engaged in the weight loss portion, the paradigm reverts to block 610 and continues from there.

However, if at block 630, it is determined that weight management participant is not engaged in the weight loss portion of the weight management paradigm, the paradigm proceeds to block 635 where a check is performed to determine if the weight management participant is engaged in the maintenance portion of the weight management paradigm. If the check at block 635 indicates that the weight management participant is not engaged in the maintenance portion of the weight management paradigm, the paradigm reverts to block 600 and continues there from.

In the instance, however, it is determined that the weight management participant is not engaged in the maintenance portion of the weight management paradigm, the paradigm proceeds to block 640 where a check is performed to determine if the weight management participant has already performed the recommended fasting periods for a selected period of time T′. If the check at block 640 is true, processing reverts back to the input of block 640 and continues there from. However, if the check at block 640 returns a false result, that is, the weight management participant has not performed the recommended “low intake” periods for the selected period of time T′, the weight management paradigm proceeds to block 645 where a check is performed to ascertain if the weight management paradigm is within the selected period of time T1. If the check at block 645 indicates that the weight management paradigm is within the selected period of time T1, the paradigm proceeds to block 650 where the paradigm directs the weight management participant to consume a first selected percentage of a total caloric intake. From there, the paradigm directs the participant to chronicle the caloric intake at block 655. The paradigm then reverts to block 640 and continues from there.

However, if at block 645 it is determined that the paradigm is not within the selected time period T1, it is presumed that the paradigm is within the selected time period T2 (not shown) and the participant is directed to intake a second selected percentage of the total caloric intake over time period T2. From there, the paradigm reverts to block 640 and continues there from.

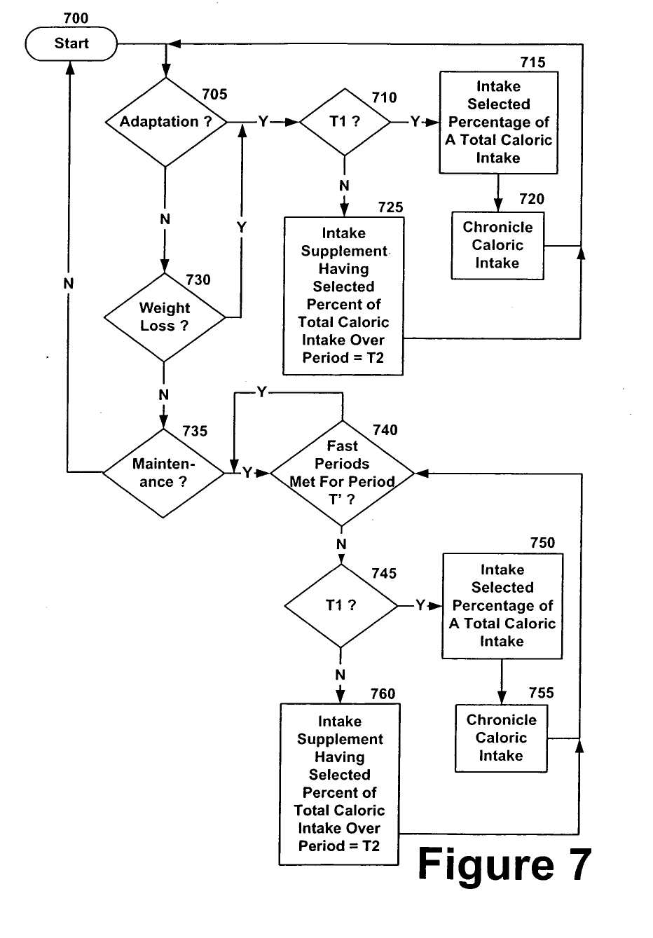

FIG. 7 shows the acts performed in an alternate implementation when applying weight management paradigm 315. As is shown, weight management paradigm 315 begins at block 700 and proceeds to block 705 where a check is performed to determine if the weight management participant is engaged in the adaptation portion of the weight management paradigm. If the weight management participant is engaged in the adaptation portion of the weight management paradigm, the paradigm proceeds to block 710 where a check is performed to determine if the weight management paradigm in within the selected time period T1. If the check at block 710 indicates that it is time period T1, the paradigm proceeds to block 715 where the participant is directed to consume a selected percentage of a total caloric intake. The paradigm then directs the participant to chronicle the caloric intake of block 715 at block 720. From there the weight management paradigm processing reverts to block 700 and continues onward.

If, however, the check at block 710 indicates that the paradigm is not within the selected time period T1, it is presumed that the paradigm is within the selected time period T2 (not shown) and the participant is directed to intake a supplement having a second selected percentage of the total caloric intake over time period T2. From there, the paradigm reverts to block 700 and continues onward.

In the instance, however, it is determined that at block 705, the weight management participant is not engaged in the adaptation portion of the weight management paradigm, the paradigm proceeds to block 730 where a check is performed to determine if the weight management participant is engaged in the weight loss portion of the weight management paradigm. If the check at block 730 indicates that the participant is engaged in the weight loss portion, the paradigm reverts to block 710 and continues from there.

However, if at block 730, it is determined that weight management participant is not engaged in the weight loss portion of the weight management paradigm, the paradigm proceeds to block 735 where a check is performed to determine if the weight management participant is engaged in the maintenance portion of the weight management paradigm. If the check at block 735 indicates that the weight management participant is not engaged in the maintenance portion of the weight management paradigm, the paradigm reverts to block 700 and continues there from.

In the instance, however, it is determined that the weight management participant is not engaged in the maintenance portion of the weight management paradigm, the paradigm proceeds to block 740 where a check is performed to determine if the weight management participant has already performed the recommended “low intake” periods for a selected period of time T′. If the check at block 740 is true, processing reverts back to the input of block 740 and continues there from. However, if the check at block 740 returns a false result, that is, the weight management participant has not performed the recommended fasting periods for the selected period of time T′, the weight management paradigm proceeds to block 745 where a check is performed to ascertain if the weight management paradigm is within the selected period of time T1. If the check at block 745 indicates that the weight management paradigm is within the selected period of time T1, the paradigm proceeds to block 750 where the paradigm directs the weight management participant to consume a first selected percentage of a total caloric intake. From there, the paradigm directs the participant to chronicle the caloric intake at block 755. The paradigm then reverts to block 740 and continues from there.

However, if at block 745 it is determined that the paradigm is not within the selected time period T1, it is presumed that the paradigm is within the selected time period T2 (not shown) and the participant is directed to intake a supplement having a second selected percentage of the total caloric intake over time period T2. From there, the paradigm reverts to block 740 and continues there from.

Health Benefits Related to Following Weight Management Paradigm

Beyond weight management in humans, the process described herein is believed to offer other health benefits. As mentioned above, animal studies suggest that CR may contribute to extending the subject's lifespan by triggering one or more genetic markers that are believed to have a direct effect on longevity. The CR program described above is also believed to provide beneficial health effects by modifying the body's reaction to stimuli that cause certain diseases and chronic conditions. These include arthritis (both osteoarthritis and rheumatoid arthritis), bursitis, asthma and bronchitis, allergies, and other inflammatory conditions. Following the CR process of the invention also yields a marked reduction in insulin resistance, which can ameliorate or avoid non-insulin dependant diabetes (NIDDM). The process also can reduce the complications of insulin and non-insulin dependent diabetes, including neuropathy, nephropathy, retinopathy, angiopathy and associated large vessel atherosclerosis.

1. A method for accomplishing human weight management comprising:
(a) providing a selected percentage of a total caloric intake during a first time period T1; and
(b) providing a second selected percentage of the total caloric intake during a second time period T2.

2. The method as recited in claim 1 further comprising providing support so that the first and second selected percentages of the total caloric intake are adhered to.

3. The method as recited in claim 1 further comprising providing a value for the total caloric intake from one or more dietary guidelines.

4. The method as recited in claim 1 further comprising providing the second selected percentage of the total caloric intake in the form of a liquid supplement.

5. The method as recited in claim 4 further comprising providing a liquid supplement having a low content of carbohydrates and a high content of protein.

6. The method as recited in claim 1 further comprising providing a value for T2 being greater than or equal to a value for T1.

7. The method as recited in claim 1 further comprising repeating steps (a) and (b).

8. The method as recited in claim 1 further comprising providing a 100% of the total caloric intake during time period T1.

9. The method as recited in claim 1 further comprising providing a selected percentage in the range of about 10%-40% of the total caloric intake during time period T2.

10. The method as recited in claim 1 further comprising chronicling the total caloric intake in a journal.

11. The method as recited in claim 1 further comprising repeating steps (a) and (b) for a selected number of times.

12. The method as recited in claim 1 further comprising providing counseling regarding caloric intake.

13. A method for accomplishing human weight management comprising:
intaking a first selected percentage of a total calorie value during a period of time T1; and
intaking a second selected percentage of the total calorie value during a second time period of time T2.

14. The method as recited in claim 13 further comprising obtaining support so that the first and second percentages of the total calorie value are adhered to.

15. The method as recited in claim 13 further comprising obtaining the total calories value from one or more dietary guidelines

16. The method as recited in claim 13 further comprising obtaining the second selected percentage of the total calories from a liquid supplement.

17. The method as recited in claim 13 further comprising obtaining a liquid supplement having a low content of carbohydrates and high content of protein.

18. The method as recited in claim 13 further comprising setting a value for T2 being greater than or equal to a value for T1.

19. The method as recited in claim 13 further comprising repeating steps (a) and (b).

20. The method as recited in claim 13 further comprising intaking 100% of the total calories during time period T1.

21. The method as recited in claim 13 further comprising intaking a selected percentage in the range of about 10%-50% of the total caloric intake during time period T2.

22. The method as recited in claim 13 further comprising chronicling the total calorie intake in a journal.

23. The method as recited in claim 13 further comprising repeating steps (a) and (b) for a selected number of times.

24. The method as recited in claim 13 further comprising obtaining counseling regarding calorie intake.

25. A system for human weight management comprising:
first means for tracking varying time periods; and
second means to identify caloric intake during the varying time periods such that the caloric intake during a first time period is greater of a caloric intake during a second time period.

26. The system as recited in claim 25 further comprising a third means for chronicling the caloric intake during the varying time periods.

27. The system as recited in claim 25 wherein the average intake over the first and second time periods is a selected percentage in the range of about 50-70% of a total caloric intake.

28. The system as recited in claim 25 further comprising a fourth means for monitoring the caloric intake over the varying time periods, wherein the caloric intake is performed on a periodic or semi-periodic basis over the varying time periods.

29. A dietary process for achieving reduction of the severity and incidence of inflammatory conditions in humans, including arthritis, asthma, bursitis, and atherosclerosis, comprising:
(a) providing a selected percentage of a total caloric intake during a first time period T1; and
(b) providing a second selected percentage of the total caloric intake during a second time period T2.

30. The method as recited in claim 29 further comprising providing a value for the total caloric intake from one or more dietary guidelines.

31. The method as recited in claim 29 further comprising providing a value for T2 being greater than or equal to a value for T1.

32. The method as recited in claim 29 further comprising repeating steps (a) and (b).

33. The method as recited in claim 29 further comprising providing a 100% of the total caloric intake during time period T1.

34. The method as recited in claim 29 further comprising providing a selected percentage in the range of about 10%-40% of the total caloric intake during time period T2.

35. The method as recited in claim 29 further comprising repeating steps (a) and (b) for a selected number of times.

36. A dietary process for achieving reduction of the severity and incidence of complications resulting from both insulin dependent and non-insulin dependent diabetes, comprising:
(a) providing a selected percentage of a total caloric intake during a first time period T1; and
(b) providing a second selected percentage of the total caloric intake during a second time period T2.

37. The method as recited in claim 36 further comprising providing a value for the total caloric intake from one or more dietary guidelines.

38. The method as recited in claim 36 further comprising providing a value for T2 being greater than or equal to a value for T1.

39. The method as recited in claim 36 further comprising repeating steps (a) and (b).

40. The method as recited in claim 36 further comprising providing a 100% of the total caloric intake during time period T1.

41. The method as recited in claim 36 further comprising providing a selected percentage in the range of about 10%-40% of the total caloric intake during time period T2.

42. The method as recited in claim 36 further comprising repeating steps (a) and (b) for a selected number of times.

43. A dietary process for activating genetic effects that contribute to the longevity of humans such that lifespan is increased, comprising:
(a) providing a selected percentage of a total caloric intake during a first time period T1; and
(b) providing a second selected percentage of the total caloric intake during a second time period T2;
(c) providing a value for the total caloric intake from one or more dietary guidelines;
(d) providing a value for T2 being greater than or equal to a value for T1;
(e) repeating steps (a) and (b);
(f) providing a 100% of the total caloric intake during time period T1;
(g) providing a selected percentage in the range of about 10%-40% of the total caloric intake during time period T2.

44. The method as recited in claim 43 further comprising repeating steps (a) and (b) for a selected number of times.

45. The method of claim 43 further comprising repeating steps (a) through (g) throughout a person's lifetime.
